(19) United States
(12) Patent Application Publication
(10) Pub. No.: US 2008/0293127 A1
(43) Pub. Date: Nov. 27, 2008

(54) Method to Render Pressure Sensitive Adhesive Compatible with Polymerase Chain Reaction Systems

(75) Inventors: Yuh-Min Chiang, Foster City, CA (US); Aldrich N.K. Lau, Palo Alto, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/752,243

(22) Filed: May 22, 2007

(51) Int. Cl. C08J 3/00; C12M 1/00

(52) U.S. Cl. 435/283.1; 528/503

(57) ABSTRACT

A method for substantially eliminating a cross-linking element of a pressure sensitive adhesive by heating the pressure sensitive adhesive at a temperature of at least 180° C. for a period of time required to thermally decompose the cross-linking element of the pressure sensitive adhesive.

### CROSS-REFERENCE TO CO-PENDING APPLICATIONS

All literature and similar materials cited in this application, including but not limited to, patents, patent applications, articles, books, treatises, and internet web pages, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

### FIELD

This disclosure is directed to rendering a pressure sensitive adhesive compatible with polymerase chain reaction systems. More particularly, the disclosure is directed to the break down and hence elimination of residual cross-linking elements of pressure sensitive adhesives in order to avoid interference with and/or inhibition of reference dyes in polymerase chain reaction (PCR) systems.

### BACKGROUND

Pressure sensitive adhesives (PSA) adhere to most surfaces with very slight pressure. They are available in a wide variety of chemical compositions or systems. Some of the most common types of systems include acrylate and methacrylate adhesives comprising, for example, ethylene-ethylacrylate copolymer (EEA) or ethylene-methylacrylate copolymer (EMA); rubber-based pressure sensitive adhesives comprising, for example, polyurethane, chloroprene, butyl, polybutadiene, isoprene or neoprene; styrene copolymers comprising, for example, styrene-isoprene-styrene copolymer (SIS) and styrene-butadiene-styrene copolymer (SBS) copolymers; and silicone adhesives comprising, for example, poly(dimethylsiloxane), poly(methylphenylsiloxane), poly(diphenylsiloxane), and their copolymers. Pressure sensitive adhesives are designed with a balance between flow and resistance to flow. The bond forms because the adhesive is soft enough to flow, or wet, the surface of an adherend. The bond has strength because the adhesive is hard enough to resist flow when stress is applied to the bond. In order to improve the bond strength, the base polymer of a pressure sensitive adhesive is commonly crosslinked by chemical means after they are coated on a backing. Chemical crosslinking can be induced by radiation, including, for example, radiation by ultra-violet light and electron beam radiation. Chemical crosslinking can also be induced by free radicals produced therein by photo or thermal decomposition of organic or organo-metallic compounds. The said organic compounds can include, but are not limited to, peroxides, for example, t-butyl hydroperoxide, benzoyl peroxide, di-t-butyl peroxide, t-butyl cumyl peroxide, and dicumyl peroxide; peroxy-esters, for example, t-butyl peroxyneodecanoate, t-butyl peroxybenzoate; peroxydicarbonates, for example, di(2-ethylhexyl)peroxydicarbonate, bis(4-t-butylcyclohexyl) peroxydicarbonate, and other peroxy compounds; and azo compounds, for example, 2,2′-azobis(2-methylbutyronitrite), 2,2′-azobis[2-methyl-N-(2-hydroxyethyl)propionamide, and 2,2′-azobis[N-(2-propenyl)-2-methylpropionamid.

Pressure sensitive adhesives can be used in combination with a film or cover member to seal an assay within an assay support structure prior to processing of the assay in a polymerase chain reaction (PCR) system.

Accordingly, the pressure sensitive adhesive should be chemically compatible with components of the assay, particularly those of a PCR master mix and nucleic acids utilized in the assay. The PCR master mix typically includes reagents, enzymes, buffers, primers and probes such as fluorescent dyes. More specifically, the PCR master mix typically utilizes a reference dye therein.

An exemplary pressure sensitive adhesive used to adhere a film or cover member to the assay support can be a silicone-based pressure sensitive adhesive. It is known that silicone exhibits superior thermal and chemical resistance during thermal cycling in the PCR system However, pressure sensitive adhesives are typically crosslinked by a crosslinking agent, including but not limited to peroxide, for example, benzoyl peroxide, to improve bond strength and high temperature performance.

For those of ordinary skill in the art, it will also be appreciated that crosslinking in general can also be conducted under photo radiation, for example, UV radiation in the presence of a photo-initiator. Commonly used photo-initiators are mainly carbonyl-containing organic compounds, including, but not limited to, benzophenone, Michler's ketone, ketocoumarins, benzoin ethers, ethyl 4-(dimethyamino)benzoate, 4,4′-bis(dimethylamino)benzophenone, 2,2-dimethoxy-2-phenylacetophenone, 2,4,6-trimethylbenzoyidiphenylphosphine oxide, or combinations thereof. After photo-initiated crosslinking, residual initiator(s) can be decomposed by photo-bleaching, a post-treatment of prolonged UV radiation. It will be appreciated by those of ordinary skill in the art that “photo-initiator”, “photo-initiated crosslinker”, and photo crosslinker are interchangeable terminology.

It is a discovery of the present disclosure that the cross-linking element of peroxide used in pressure sensitive adhesives will interfere with and/or inhibit the reference dye typically used in the PCR master mix, resulting in the decay of fluorescent signals. This reference dye includes, but is not limited to, 6-carboxy-X-rhodamine (ROX), commonly used as a passive reference dye for normalization of a reporter signal in a PCR system.

It is therefore an exemplary object of the disclosure to eliminate a residual cross-linking element in the pressure sensitive adhesive prior to its typical application of sealing an assay in the assay support structure and introduction into the PCR system. Further, it is an exemplary object of the disclosure to eliminate the residual cross-linking element in the pressure sensitive adhesive by decomposing the cross-linking element, such as by thermal decomposition or photo-bleaching. The present disclosure is further novel in that the process does not add an additional reagent to neutralize or physically remove anything from either the PCR master mix or the pressure sensitive adhesive. As a result of the thermal treatment or photo-bleaching, the concentration of residual crosslinking agent becomes so low that signal decay in PCR is not observed. In addition, the pressure sensitive adhesive having a low residual volatile can be especially advantageous for those assays containing a dried form of reagent therein for use at a later time.

Accordingly, the present disclosure provides a novel solution for eliminating the cross-linking residue in the pressure sensitive adhesive without changing existing components.

### SUMMARY

Exemplary embodiments according to aspects of the present disclosure may satisfy one or more of the desirable features set forth above. Other features and advantages will become apparent from the detailed description which follows.

In accordance with various exemplary aspects, the disclosure can include a method for eliminating a residual cross-linking element of a pressure sensitive adhesive, comprising heating the pressure sensitive adhesive at a temperature of at least about 50° C., preferably about 100° C., further preferably about 170° C., and most preferably about 180° C. for a period of time defined by a substantial decomposition of the cross-linking element used. It will be appreciated that peroxide, for example benzoyl peroxide, one of the common peroxides used in the preparation of pressure sensitive adhesives, has a half-life of 10 hours at a temperature of 78° C. Over 98% of it decomposes after heating at 180° C. for less than 10 seconds. Other peroxides have 10 hours half-life temperatures ranging from 30 to 150° C. Typically, the pressure sensitive adhesive can be a silicone-based pressure sensitive adhesive cross-linked with benzoyl peroxide. The thermal treatment for reduction/elimination of benzoyl peroxide can be 180° C.

It will be further appreciated that a cross-linker is used to prepare pressure sensitive adhesives in order to improve bond strength. During manufacturing, a mixture comprising, for example, a thermal cross-linker and the base polymer, for example, a silicone-based polymer, is heated at an elevated temperature to affect the crosslinking. Since no organic reactions give 100% yield (or 100% conversion), there is always a residual amount of cross-linker left behind, ranging from a few ppb to 1000's ppm. One of the objectives of the present invention is to eliminate (or reduce) this residual cross-linker.

In various exemplary aspects of the disclosure, the cross-linking element may be up to about 5 wt % benzoyl peroxide, up to about 3 wt % benzoyl peroxide, up to about 1 wt % benzoyl peroxide, or up to about 0.5 wt % benzoyl peroxide.

An exemplary period of time for thermally treating the pressure sensitive adhesive can be from less than 60 seconds up to two minutes depending on the temperature being used. Thermal crosslinking agents are commonly free radical initiators that decompose at an elevated temperature to give free radicals. The rate of decomposition is expressed as the time to decompose half of the initial amount at a given temperature. It is called the half-life temperature; the higher the temperature, the short the half-life. The half-life temperatures of various thermal crosslinkers, such as peroxides, are reported in published literature. The half-life for benzoyl peroxide is about 5 seconds at 180° C.

In accordance with yet other exemplary embodiments, a method for rendering a pressure sensitive adhesive compatible with components in a polymerase chain reaction (PCR) system, comprises providing an assay support structure and a sealing member for the assay support structure; introducing an assay into the assay support structure, the assay including a reference dye; applying a pressure sensitive adhesive to the sealing member; thermally treating the combined sealing member and pressure sensitive adhesive at a temperature suitable for thermally decomposing the residual cross-linking element of the pressure sensitive adhesive; sealing the assay within the assay support structure; and processing the sealed assay in the PCR system. Sealing can be prior or subsequent to thermally treating the pressure sensitive adhesive, as long as the thermal treatment is conducted without the existence of reference dyes and or other types of dyes.

In the exemplary method above, the pressure sensitive adhesive can comprise a silicone based pressure sensitive adhesive and can include benzoyl peroxide as a residual cross-linking element. Further, the step of thermally treating can be for a predetermined time defined by substantial elimination of residual benzoyl peroxide in the silicone-based pressure sensitive adhesive.

In yet another exemplary embodiment of the disclosure, a method for substantially eliminating a cross-linking element in a pressure sensitive adhesive for use in a polymerase chain reaction (PCR) system comprises providing an assay support structure and a pressure sensitive adhesive sealing member for the assay support structure; introducing an assay into the assay support structure, the assay including a reference dye; applying a pressure sensitive adhesive sealing member; thermally treating the combined sealing member and silicone-based pressure sensitive adhesive at a temperature of about 180° C. for a period of time defined by substantial elimination of the residual cross-linking element; sealing the assay within the assay support structure with the sealing member; and processing the sealed assay in the PCR system. A temperature for thermally treating the combined silicone and pressure sensitive adhesive can be at a lower temperature but with a correspondingly extended period of time to thermally decompose the residual cross-linker of the adhesive.

In a further exemplary embodiment of the disclosure, there is provided an assay card for use in a polymerase chain reaction system. The assay card includes an assay support structure; a master mix and assay disposed in the support structure; a sealing film coated with a pressure sensitive adhesive applicable to the support structure; and the pressure sensitive adhesive being treated at a temperature of at least about 180° C. for a predetermined period of time and prior to using the sealing film in the assay support structure.

The pressure sensitive adhesive typically can include a cross-linking component of benzoyl peroxide residue at low concentration and the predetermined period of time is defined by a time required to substantially eliminate the residual benzoyl peroxide in the pressure sensitive adhesive.

In the following description, certain aspects and embodiments will become evident. It should be understood that the disclosure, in its broadest sense, could be practiced without having one or more features of these aspects and embodiments. It should be understood that these aspects and embodiments are merely exemplary and explanatory and are not restrictive of the disclosure.

### BRIEF DESCRIPTION OF THE DRAWINGS

The drawings of this application illustrate exemplary embodiments of the disclosure and together with the description, serve to explain certain principles. In the drawings:

FIG. 1 is a multi-component graph illustrating the decay of fluorescent signal of a reference dye over a period of time, in the presence of selected concentrations of a cross-linking component;

FIG. 2 is a multi-component graph illustrating a reference signal output from an assay card in a PCR system, the assay card including a silicone-based pressure sensitive adhesive thermally treated at about 180° C. over a period of time;

FIG. 3 is a multi-component graph illustrating a reference signal output from an assay card in a PCR system, the assay card including a silicone-based pressure sensitive adhesive thermally treated at about 140° C. over a period of time;

FIG. 4 is a top perspective view illustrating a typical assay support structure for use with some embodiments of the present teachings;

FIG. 5 is a side view illustrating an assay support structure in accordance with some embodiments of the present teachings;

FIG. 6 is a cross-sectional view illustrating an assay support structure employing a plurality of sample wells, and a sealing cover according to some embodiments of the present teachings;

FIG. 7 is a cross-sectional view illustrating details of a sealing cover according to some embodiments of the present teachings; and

FIG. 8 is an exploded perspective view illustrating an exemplary assay card for use according to some embodiments of the present teachings.

### DESCRIPTION OF VARIOUS EMBODIMENTS

In this application, the use of singular includes the plural unless specifically stated otherwise. In this application, the use of “or” means “and/or” unless stated otherwise. Furthermore, the use of the term “including”, as well as other forms, such as “includes” and “included”, is not limiting. Wherever possible, the same reference numbers will be used throughout the drawings to refer to the same or like parts.

The section headings used herein are for organizational purposes only, and are not intended to be construed as limiting the subject matter described. All documents cited in this application, including, but not limited to patents, patent applications, articles, books, treatises, are expressly incorporated by reference in their entirety for any purpose. In the event that one or more of the incorporated literature and similar materials differs from or contradicts this application, including but not limited to defined terms, term usage, described techniques, or the like, this application controls.

When referring to various directional relationships herein, such as, for example, downward, upward, left, right, top, bottom, etc., such relationships are referred to in the context of the orientation of the drawings, unless otherwise specified. It should be understood however, that the devices in actuality may be oriented in a direction other than those illustrated in the drawings and directional relationships would vary accordingly.

The term assay, as used herein, refers to chemicals including enzymes that are designed for a specific sample used in PCR reactions.

The term polymerase chain reaction (PCR), as used herein, refers to a technique used to amplify the number of copies of a specific region of DNA, in order to produce enough DNA to be adequately tested.

The term master mix, as used herein, typically comprises reagents, enzymes, buffers, primers and probes, and fluorescent dyes.

The term ROX, as used herein, refers to a “passive” reference dye commonly used in Applied Biosystems' PCR product (for normalization of reporter signal) and has a chemical composition of 6-carboxy-X-rhodamine.

The term reference dye, as used herein, refers to a dye utilized in all reactions to obtain normalized reporter signal (Rn) adjusted for well-to-well variations by the analysis software. The most common reference dye is ROX and is usually included in the master mix.

The term thermal cycling, as used herein, refers to a reaction process in a PCR system in which the assay and master mix are heat processed.

The terms “signal degradation” and “signal decay”, as used herein, refer to a detected fluorescent signal output from a reference dye in a master mix during thermal cycling of the PCR system and the signal's declining output over a period of time.

The term “eliminate” or “elimination” with respect to thermal decomposition of a cross-linking element in a pressure sensitive adhesive, as used herein, refers to a concentration of the cross-linker being rendered so low that it no longer interferes with and/or degrade the signal of a reference dye in a PCR system.

The term “reduction” and “reducing”, as used herein, can be used interchangeably with the terms “eliminate” or “eliminating”.

Reference will now be made to various embodiments, examples of which are illustrated in the accompanying drawings. However, it will be understood that these various embodiments are not intended to limit the disclosure. On the contrary, the disclosure is intended to cover alternatives, modifications, and equivalents.

Although the present teachings will be discussed in some embodiments as relating to polynucleotide amplification, such as PCR, such discussion should not be regarded as limiting the present teaching to only such applications.

A typical polymerase chain reaction (PCR) system can include an assay card, a microplate, a next generation micro card, or any other similar card having an assay master mix associated therewith into a PCR processing system. These known cards and plates are typically sealed with a film having a pressure sensitive adhesive on at least one side thereof. The film seals all components, including the master mix materials within the card for processing in the PCR system.

The master mix utilized in many assays typically can include a reference dye therein. The reference dye can exhibit normalized reporter signals adjusted for well-to-well variations by analysis software in a reaction initiated in the PCR system. One of the common reference dyes found in a typical master mix is 6-carboxy-X-rhodamine (ROX). In addition, a dried master mix can be pre-deposited in a reaction well with the micro card being stored for a period of time prior running a PCR.

The pressure sensitive adhesive sealing film can be used due to its applicability to many varied assay structures. In order to withstand temperatures and processing of the PCR system, silicone-based pressure sensitive adhesives can be used. These silicone-based pressure sensitive adhesives are typically cross-linked by a cross-linking element for improving bond strength and physical properties of the adhesive. A common cross-linking element in the manufacturing of silicone-based pressure sensitive adhesive is benzoyl peroxide. In a silicone-based pressure sensitive adhesive manufactured by Adhesive Research, Inc., about 5% by weight of benzoyl peroxide was used for cross-linking during the manufacturing. Other percentages of benzoyl peroxide can be used in the manufacturing to cross-link the base polymer. For example, about 0.5 wt %, 1 wt %, or 3 wt % of benzoyl peroxide can be used in the preparation/manufacturing of pressure sensitive adhesives, according to the type of adhesive supplied.

However, it is a discovery of the present disclosure that the silicone-based PSA (manufactured by Adhesive Research, Inc.), can cause fluorescent signal decay in the master mix during the thermal cycling of the PCR process. It is a further discovery of the present disclosure that the silicone-based PSA more specifically can cause fluorescent signal decay of the reference dye (ROX) in the master mix. It is yet another discovery of the present disclosure that the benzoyl peroxide used as a cross-linking element in the silicone-based PSA manufactured by Adhesive Research, Inc. can cause fluorescent signal decay of the reference dye in the master mix, thus compromising the processing results of the PCR system.

In theory, benzoyl peroxide can decompose to form free radicals at temperatures above about 50 C. The free radicals can then render crosslinking of the base polymer(s) in the pressure sensitive adhesive. However, benzoyl peroxide residue can be left in the adhesive, due to incomplete decomposition, resulting in undesirable interference with the reference dye ROX when the PSA is used in a PCR system.

Accordingly, the present disclosure can solve the problem of signal decay of the reference dye in a PCR system as discovered by the inventors. In an exemplary embodiment disclosed herein, residual benzoyl peroxide in the silicone-based PSA can be broken down (decomposed) and eliminated as a potential interference in the PCR system.

This break down and hence elimination of residual benzoyl peroxide can be achieved by thermally treating the silicone-based PSA at a predetermined temperature for a predetermined period of time before the silicone-based PSA is used in an assay card.

In an exemplary embodiment of the disclosure, initial preparation of a silicone-based pressure sensitive adhesive with a benzoyl peroxide cross-linker can be obtained from Adhesive Research, Inc. The present disclosure can then include a thermal treatment step in which the silicone-based PSA can be thermally treated (baked) at a temperature of at least about 180° C. for a predetermined period of time. This predetermined period of time can range from seconds to minutes depending upon the temperature of the thermal processing and thickness of the silicon-based pressure sensitive adhesive. Attached test results confirm thermal decomposition of the cross-linker at a temperature up to 200° C.

Thermal treatment of the silicone-based pressure sensitive adhesive as described will eliminate residual benzoyl peroxide cross-linker from the adhesive, thus preventing its interference with the reference dye during processing or thermal cycling in a PCR system.

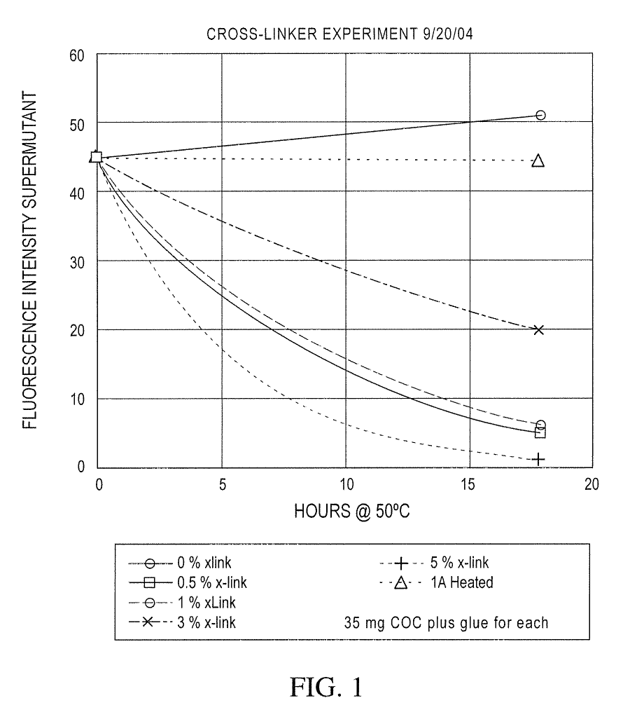

Supporting experimental results of the exemplary embodiments are particularly shown in FIGS. 1 through 3.

Referring first to FIG. 1, there is illustrated exemplary fluorescent signal decay of the reference dye ROX with various concentrations of benzoyl peroxide used in preparing the PSA samples. Specifically, test results are shown for the cross-linking element benzoyl peroxide at a percentage by weight at each of about 0%, 0.5%, 1%, 3%, and 5% concentration of cross-linking element to master mix.

In each sample, there can be included about 35 mg of cyclic olefin copolymer (COC) backing material coated with a silicone-based pressure sensitive adhesive. The sample was soaked in a master mix comprising ROX as the reference dye. The results indicate fluorescence intensity in the supernatant measured over approximately 18 hours at about 50° C. The sample prepared with 0% of the cross-linking element showed no signs of fluorescent signal decay over the entire duration of testing, while the remaining samples showed progressively increasing signal decay over the same time duration. Clearly, it can be appreciated that the fluorescent signal decay of ROX is due to the presence of the benzoyl peroxide residues within the sample of silicone-based pressure sensitive adhesive.

Accordingly, at the time of preparing a silicone-based pressure sensitive adhesive for laminating or sealing an assay card, the adhesive can be thermally treated at a final processing temperature of at least about 180° C. for a predetermined period of time. The predetermined period of time is that which is sufficient to eliminate the residual cross-linking element from the pressure sensitive adhesive. For example, the predetermined time can be about 20 minutes. The predetermined time can be, for example, less than 10 minutes. For example, the predetermined time can be 30 seconds at 200° C. or 1 minute at 180° C. This temperature of about 180° C. can be high enough to decompose residual benzoyl peroxide within the silicone-based PSA. Test results show no decay of ROX signal over time with the thermal treatment described. The thermal treatment of the pressure sensitive adhesive typically can occur subsequent to application of the adhesive to the cover member. However, the present disclosure is not intended to exclude thermal treatment prior to application of the adhesive to the cover member.

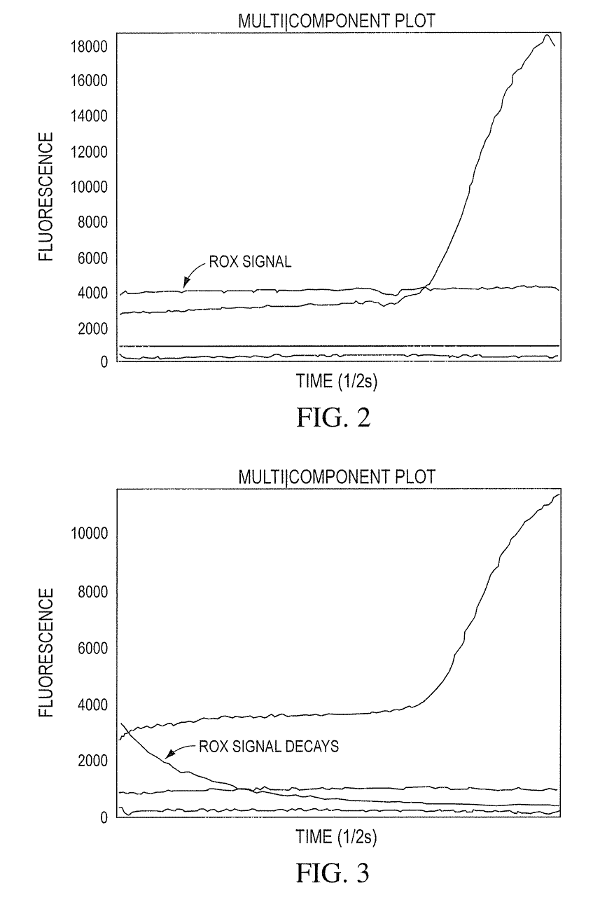

Referring next to FIG. 2, it is seen that a reporter signal emitted by the reference dye ROX is constant over time when the silicone-based PSA was thermally treated in a final processing step of at least about 180° C.

To the contrary, and referring to test results of FIG. 3, a reporter signal emitted by the reference dye ROX will decay over time when the silicone-based PSA is thermally treated in a final processing step of only about 140° C. Even further, the following data reveals that there is no decay of the ROX signal, even after being stored for three months with a dried master mix.

In order to demonstrate the continuity of signal intensity in samples whose residual cross-linking element has been thermally decomposed, the following Experimental results are provided. Properties of sample pressure sensitive adhesives are depicted in the following:

PSA PROPERTY CHART
PSA Property

| | | Temp | Temp | Total | | | | |
|---|---|---|---|---|---|---|---|---|
| Sample ID | Construction | Zone 1 | Zone 2 | organics | Siloxanes | Hydrocarbons | Alcohols | Others |
| Trial roll | 4.7 mil Soft Foil/1.8 mil SR-26 | N/A | 170 C. | 1463.16 | 748.75 | — | 319.48 | 394.93 |
| Condition 2 | 4.7 mil Rigid Foil/1.8 mil SR-26 | 150 C. | 200 C. | 100.46 | 14.55 | 7.73 | 35.75 | 42.42 |
| Condition 7 | 4.7 mil Rigid Foil/1.8 mil SR-26 | 180 C. | 200 C. | 88.6 | 1.27 | 15.9 | 48.94 | 22.48 |
| Condition 9 | 4.7 mil Rigid Foil/1.8 mil SR-26 | 180 C. | 200 C. | 94.91 | 0.81 | 19.98 | 53.16 | 20.95 |
| MH-90880 | 1 mil SR-26/2 mil BOPP/1 mil SR-26 | N/A | 180 C. | 373.32 | 17.28 | 4.76 | 207.36 | 143.92 |

* PSA prepraed by H Coater
* Outgassing unit is ppm

An exemplary assay card preparation can include an assay and template of 10K Flu A. There can be 24 sample wells per card, with 12 wells containing the reference dye ROX and 12 wells containing the reference dye Mustang Purple (MP). The PCR can be run on an AB7900 manufactured by Applied Biosystems. Results are taken at Day 1 and Month 1, shown below.

DAY 1 RESULTS CHART

| | Rox | | | | | | MP | | | | | |
|---|---|---|---|---|---|---|---|---|---|---|---|---|
| Al | | | | | Avg | | | | | | Avg | |
| PSA | initial | iH-L | final | % | Ct | StdEv | initial | iH-L | final | % | Ct | StdEv |
| Trial | 2518 | 800 | 2518 | 0 | 27.41 | 0.11 | 2125 | 1000 | 1933 | −9 | 26.40 | 0.26 |
| (1) | | | | | | | | | | | | |
| Trial | 2500 | 1300 | 2473 | −1 | 26.03 | 0.09 | 2258 | 500 | 2033 | −10 | 25.97 | 0.80 |
| (2) | | | | | | | | | | | | |
| # 2 (1) | 3250 | 2400 | 3233 | −1 | 28.50 | 0.31 | 2250 | 900 | 2050 | −9 | 26.44 | 0.26 |
| # 2 (2) | 3000 | 1600 | 2864 | −4 | 27.32 | 0.89 | 2682 | 1000 | 2555 | −4 | 29.89 | 1.90 |
| # 7 (1) | 2573 | 950 | 2498 | −3 | 28.30 | 0.20 | 1979 | 500 | 1908 | −3 | 29.06 | 0.90 |
| # 7 (2) | 2625 | 900 | 2617 | 0 | 26.81 | 0.10 | 2333 | 800 | 2175 | −7 | 27.76 | 0.98 |
| # 9 (1) | 2513 | 480 | 2380 | −5 | 28.28 | 0.20 | 2050 | 700 | 1933 | −5 | 27.95 | 1.24 |
| # 9 (2) | 2860 | 1200 | 2775 | −3 | 27.00 | 0.15 | 2230 | 900 | 2090 | −6 | 27.97 | 1.10 |

MONTH 1 RESULTS CHART

| | Rox | | | | | | MP | | | | |
|---|---|---|---|---|---|---|---|---|---|---|---|
| Al PSA | initial | iH-L | final | % | Avg. Ct | StdEv | initial | iH-L | final | % | Avg.Ct |
| Trial | 2458 | 1000 | 2458 | −0.3 | 26.97 | 0.12 | 2092 | 1000 | 1958 | −6.5 | — |
| (1) | | | | | | | | | | | |
| Trial | 2382 | 800 | 2345 | −1.9 | 27.60 | 0.23 | 2088 | 1250 | 1931 | −7.9 | — |
| (2) | | | | | | | | | | | |
| # 2 (1) | 2618 | 900 | 2582 | −1.4 | 28.89 | 0.29 | 2100 | 800 | 1991 | −5.3 | — |
| # 2 (2) | 2567 | 800 | 2454 | −4.4 | 28.75 | 0.19 | 1964 | 1000 | 1809 | −7.8 | — |
| # 7 (1) | 2533 | 700 | 2550 | 0.7 | 27.77 | 0.12 | 2045 | 650 | 1955 | −4.3 | — |
| # 7 (2) | 2645 | 900 | 2573 | −2.8 | 27.82 | 0.15 | 2054 | 1100 | 1942 | −5.6 | — |
| # 9 (1) | 2375 | 1200 | 2383 | 0.1 | 29.16 | 0.17 | 2008 | 900 | 2008 | −0.2 | — |
| # 9 (2) | 2527 | 900 | 2518 | −0.2 | 29.10 | 0.16 | 2150 | 600 | 2142 | 0.0 | — |

Each of the Control, Condition #2, Condition #7 and Condition #9 with the reference dye ROX are graphically indicated in the following. It can be seen that the output signal of the reference dye remains constant, i.e. does not decay at room temperature (RT), even at one month.

Each of the Control, Condition #2, Condition #7, and Condition #9 with the reference dye ROX is graphically indicated in the following. It can be seen that the output signal of the reference dye remains constant, i.e. does not decay at 45° C., even at one month.

Each of the Control, Condition #2, Condition #7 and Condition #9 with the reference dye MP are graphically indicated in the following. It can be seen that the output signal of the reference dye remains constant, i.e. does not decay at room temperature (RT), even at one month.

Each of the Control, Condition #2, Condition #7 and Condition #9 with the reference dye MP are graphically indicated in the following. It can be seen that the output signal of the reference dye remains constant, i.e. does not decay at 45° C., even at one month.

Condition samples #2, #7 and #9 show comparable Ct numbers in day 1, Month 1, Month 2, and Month 3, stored at room temperature, when subjected to a PCR run. Results are shown below.

It will be appreciated that the disclosed teachings can be utilized with any sealing type film or cover member having a cross-linker incorporated into the adhesive in its preparation. Examples of such adhesives and cross-linkers have been described above. It will be further appreciated that the cross-linker can include a peroxide cross-linker and that the adhesive can include a non-silicone based pressure sensitive adhesive. It will be even further appreciated that the crosslinker can include a photo-crosslinker that initiates crosslinking by photo-radiation, for example radiation by ultra violet light, and the elimination of residual photo-crosslinker can be by photo bleaching. More specifically, it will be appreciated that the disclosed exemplary teachings can be utilized in a sealing type film or cover having a benzoyl peroxide cross-linker in a silicone-based pressure sensitive adhesive used to seal an assay card or the like prior to processing of the assay in a PCR system. Examples of typical microplate or assay card structures and assay sealing members follow.

In some exemplary embodiments, an assay can comprise any material that is useful in, the subject of, a precursor to, or a product of, an analytical method or chemical reaction. In some embodiments for amplification and/or detection of polynucleotides, the assay comprises one or more reagents (such as a PCR master mix); an analyte (such as a biological sample comprising DNA, a DNA fragment, cDNA, RNA, or any other nucleic acid sequence); one or more primers; one or more primer sets; one or more detection probes; components thereof; and combinations thereof.

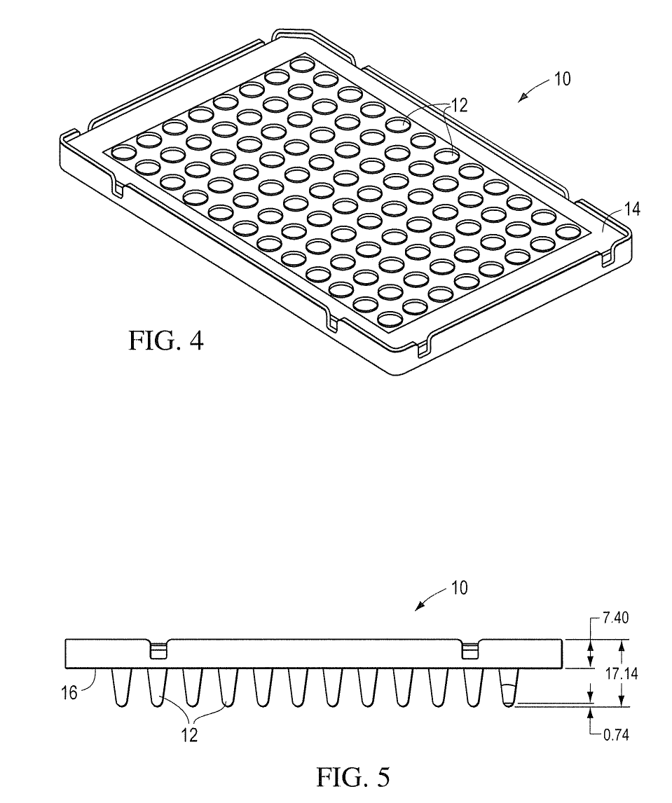

Referring first to FIG. 4, an assay card or microplate 10 is illustrated which can be incorporated in the exemplary embodiments of the invention. The microplate 10 can be used to support the assay in an analytical method or chemical reaction. In some embodiments, a microplate 10 can comprise one or more material retention regions 12, configured to hold or support the assay at one or more locations on or in the microplate 10. These material retention regions 12 are typically described as wells. The term microplate is intended to be used interchangeably with other support structure terms such as assay card and the like.

FIG. 5 is a side view of the microplate 10, more clearly illustrating the relationship of the wells 12 to the microplate 10. The microplate 10 typically can comprise a construction having an upper surface 14 and an opposing lower surface 16. Upper surface 14 includes the plurality of wells 12 disposed therein or thereon. The overall positioning of the plurality of wells 12 can be referred to as a well array. Each of the plurality of wells 12 is sized to receive the assay.

According to exemplary embodiments, and as illustrated, each of the plurality of wells 12 can be substantially equivalent in size. The plurality of wells 12 can have any cross-sectional shape. It will be appreciated that the wells can be generally circular in shape as shown in the figure, or have a generally square-shaped configuration. A particular shape of the well 12 is not critical to an understanding of the disclosure and is provided for purposes of environment and explanation.

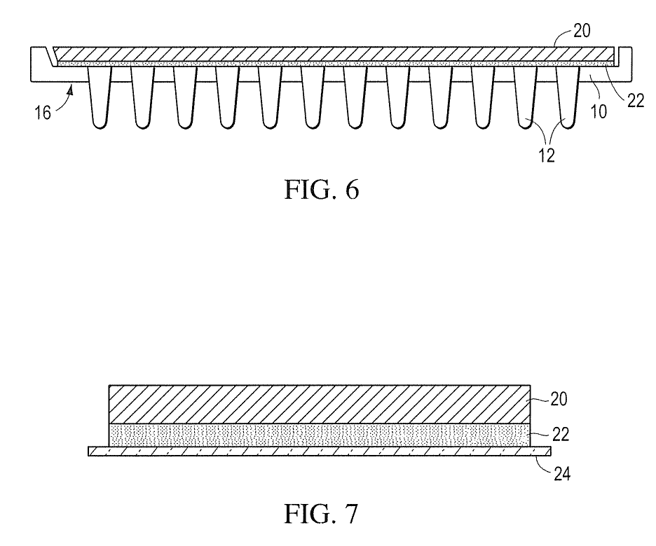

Referring to FIG. 6, a sealing cover or film 20 can be generally disposed across microplate 10 to seal the assay within each of the plurality of wells 12 of microplate 10. That is, sealing cover 20 can seal and thereby isolate each of the plurality of wells 12 from adjacent wells 12, thus maintaining sample integrity between each of the plurality of wells 12 and reducing the likelihood of cross contamination between wells. Sealing cover 20 can include a commercially available pressure sensitive tape having a layer of crosslinked pressure sensitive adhesive 22 (as depicted in FIGS. 6 and 7) coated on one side of a thin film support (i.e., sealing cover 20 in FIGS. 6 and 7). It will be further appreciated that the pressure sensitive adhesive tape can be crosslinked by a vendor prior to use in the structure of FIGS. 6 and 7. The residual benzoyl peroxide is the remnant from thermal crosslinking.

In some exemplary embodiments, sealing cover 20 can be made of any material conducive to the particular processing to be done. In some embodiments, sealing cover 20 can comprise a durable, generally optically transparent material, such as an optically clear film exhibiting abrasion resistance and low fluorescence when exposed to an excitation light.

In an exemplary embodiment, the sealing cover 20 can include the silicone-based pressure sensitive adhesive 22 on a sealing side thereof. It will be appreciated that the adhesive side 22 of the sealing film 20 can be in surface contact with the upper surface 14 (FIG. 5) of the microplate, and therefore seal the assay within each of the plurality of wells 12. By breaking down and/or eliminating the residual benzoyl peroxide in the adhesive prior to processing the assembled assay and microplate in the PCR system, contact of the adhesive side 22 of the sealing cover 20 with the assay will not degrade the reference dye of the master mix.

Referring to FIG. 7, a detailed side sectional view of the film type cover is shown, with the pressure sensitive adhesive side 22 on one side of the sealing cover 20, and a peelable backing 24 for protecting the adhesive side 22 prior to use. The peelable backing 24 may or may not be present at the time of thermally treating the adhesive 22 described above. In operation, the backing 24 may be peeled away from the adhesive side 22 of the sealing cover 20 and applied firmly across the top of the microplate 10. Once sealed, the microplate may be safely stacked or processed in the PCR system without cross-contamination of wells 12.

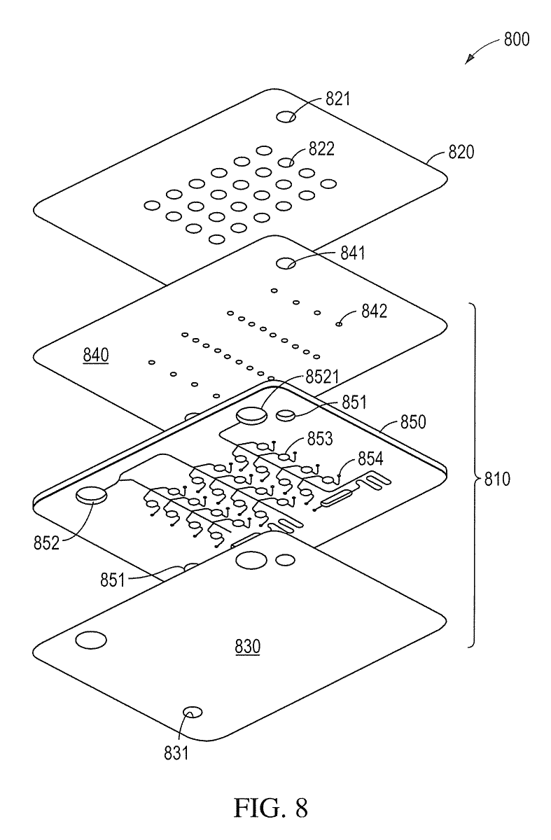

A further exemplary assay card 800 is depicted in FIG. 8. The exemplary assay card 800 can include a substrate 810 and a cover 820. Even further, the base 810 can include a metal film 830, an adhesive layer 840 and a base 850. The metal film 830 can be positioned on a lower surface of the base 850, while the adhesive layer 840 can be formed on an upper surface of the base 850. Adhesive layer 840 receives the cover 820 thereon.

The base 850 can include additional assay card components such as indexing holes 851, sample ports 852, sample chambers 853, and venting chambers 854. Metal film 830 can include indexing holes 831. Adhesive layer 840 can include indexing holes 841 and vent holes 842. Cover 820 can include indexing holes 821 and optical apertures 822 as shown. It will be appreciated that functioning of the components listed above are known, and will be omitted herein for purposes of simplicity. In keeping with the subject invention, thermal treatment of the assay card 800 herein is provided in order to thermally decompose, and therefore eliminate, any residual cross-linking agent of the adhesive layer 840. The elimination of residual cross-linking agent avoids any interference with or inhibition of reference dyes in the PCR system.

While exemplary assay cards and plates are illustrated herein, it will be appreciated that these are not intended to be limiting of the invention and in fact any support suitable for use in a PCR system can be used. It is the intent of the disclosure that the residual crosslinking components of a pressure sensitive adhesive cover (for any of these systems) be thermally treated to eliminate the residual crosslinking component therein. By elimination of the residual crosslinking element, there will be no decay of the reference dye used in a master mix of the PCR system.

It should also be understood to those having skill in the art that the various exemplary embodiments described herein may be used individually or in combination with each other. Further, the various physical modifications described herein may be used in combination with any assay support structure sealed with a silicone-based pressure sensitive adhesive and other non-silicone-based adhesives as explained above. It is additionally within the scope of the disclosure to include photo-initiated crosslinking in which at least one photo initiator is needed. In addition to the currently use of thermally crosslinked PSA, it will be appreciated that PSA can be prepared by photo-crosslinking. In this case, a system (or formulation) comprising the base polymer(s) and at least one photo initiator is first coated on a backing and then radiated under UV light to induce crosslinking. Commonly used photo initiators are mainly carbonyl-containing organic compounds, including but not limited to benzophenone, Michler's ketone, ketocoumarins, benzoin ethers, ethyl 4-(dimethyamino)benzoate, 4,4′-bis(dimethylamino)benzophenone, 2,2-dimethoxy-2-phenylacetophenone, and 2,4,6-trimethylbenzoyldiphenylphosphine oxide. After photo-initiated crosslinking, residual initiator(s), that may interfere with PCR, can be decomposed and thus substantially eliminated by post-treatment of prolonged UV radiation.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “less than 10” includes any and all subranges between (and including) the minimum value of zero and the maximum value of 10, that is, any and all subranges having a minimum value of equal to or greater than zero and a maximum value of equal to or less than 10, e.g., 1 to 5.

It is noted that, as used in this specification and the appended claims, the singular forms “a,” “an,” and “the,” include plural referents unless expressly and unequivocally limited to one referent. Thus, for example, reference to “a biological” includes two or more different biological samples. As used herein, the term “include” and its grammatical variants are intended to be non-limiting, such that recitation of items in a list is not to the exclusion of other like items that can be substituted or added to the listed items.

What is claimed is:

1. A method for eliminating a cross-linking element of a pressure sensitive adhesive, comprising:
heating the pressure sensitive adhesive at a temperature of at least 180° C. for a period of time defined by substantial elimination of said cross-linking element.

2. The method of claim 1, wherein the pressure sensitive adhesive comprises any of acrylate and methacrylate adhesives.

3. The method of claim 2, wherein the acrylate and methacrylate adhesives comprise, ethylene-ethylacrylate copolymer (EEA) or ethylene-methylacrylate copolymer (EMA); rubber-based pressure sensitive adhesives; styrene copolymers; and silicone adhesives.

4. The method of claim 3, wherein the rubber based pressure sensitive adhesives comprise polyurethane, chloroprene, butyl, polybutadiene, isoprene or neoprene.

5. The method of claim 3, wherein the styrene copolymers comprise styrene-isoprene-styrene copolymer (SIS) and styrene-butadiene-styrene copolymer (SBS) copolymers.

6. The method of claim 3, wherein the silicone adhesives comprise poly(dimethylsiloxane), poloy(methyphenylsiloxane), poly(diphenylsiloxane), and their copolymers.

7. The method of claim 1, wherein the pressure sensitive adhesive comprises a residual cross-linking element.

8. The method of claim 7, wherein the residual crosslinking element comprises photo-initiated crosslinkers.

9. The method of claim 7, wherein chemical cross-linking comprises radiation of the adhesive.

10. The method of claim 9, wherein the radiation comprises ultra-violet light and electron beam radiation.

11. The method of claim 7, wherein chemical cross-linking comprises inducing free radicals therein by photo or thermal decomposition of organic or organo-metallic compounds.

12. The method of claim 11, wherein said organic compounds comprise photo-initiated crosslinkers.

13. The method of claim 12, wherein the photo-initiated crosslinkers comprise benzophenone, Michler's ketone, ketocoumarins, benzoin ethers, ethyl 4-(dimethyamino)benzoate, 4,4′-bis(dimethylamino)benzophenone, 2,2-dimethoxy-2-phenylacetophenone, 2,4,6-trimethylbenzoyidiphenylphosphine oxide, or combinations thereof.

14. The method of claim 11, wherein said organic compounds comprise peroxides, peroxy-esters, peroxydicarbonates, and azo compounds.

15. The method of claim 11, wherein said peroxides comprise t-butyl hydroperoxide, benzoyl peroxide, di-t-butyl peroxide, t-butyl cumyl peroxide, and dicumyl peroxide.

16. The method of claim 11, wherein said peroxy-esters comprise t-butyl peroxyneodecanoate, and t-butyl peroxybenzoate.

17. The method of claim 11, wherein said peroxydicarbonates comprise di(2-ethylhexyl)peroxydicarbonate, bis(4-t-butylcyclohexyl)peroxydicarbonate, and other peroxy compounds.

18. The method of claim 11, wherein said azo compounds comprise 2,2′-azobis(2-methylbutyronitrite), 2,2′-azobis[2-methyl-N-(2-hydroxyethyl)propionamide, and 2,2′-azobis[N-(2-propenyl)-2-methylpropionamid.

19. The method of claim 1, wherein the period of time is up to 20 minutes.

20. The method of claim 19, wherein the period of time is less than 10 minutes.

21. The method of claim 19, wherein the period of time is less than 5 minutes.

22. A method for rendering a pressure sensitive adhesive compatible with components in a polymerase chain reaction (PCR) system, comprising:
providing an assay support structure and a sealing member for sealing said assay support structure;
introducing an assay into said assay support structure, said assay including a reference dye;
applying a pressure sensitive adhesive to the sealing member, the pressure sensitive adhesive including a residual cross-linking element;
thermally treating the sealing member having the pressure sensitive adhesive at a temperature and for a predetermined time suitable for thermally decomposing a cross-linking element of the pressure sensitive adhesive;
sealing the assay within the assay support structure with the sealing member; and
processing the sealed assay in the PCR system.

23. The method of claim 22, wherein the temperature is at least 180° C.

24. The method of claim 22, wherein thermally treating the sealing member is prior to sealing the assay within the assay support structure.

25. The method of claim 22, wherein thermally treating the sealing member is subsequent to sealing the assay within the assay support structure.

26. The method of claim 22, wherein the pressure sensitive adhesive comprises a silicone-based pressure sensitive adhesive.

27. The method of claim 22, wherein the residual cross-linking element comprises benzoyl peroxide.

28. The method of claim 26, wherein the residual cross-linking element comprises benzoyl peroxide.

29. The method of claim 22, wherein the residual crosslinking element comprises photo-initiated crosslinkers.

30. The method of claim 29, wherein the photo-initiated crosslinkers comprise benzophenone, Michler's ketone, ketocoumarins, benzoin ethers, ethyl 4-(dimethyamino)benzoate, 4,4′-bis(dimethylamino)benzophenone, 2,2-dimethoxy-2-phenylacetophenone, 2,4,6-trimethylbenzoyldiphenylphosphine oxide, or combinations thereof.

31. The method of claim 22, wherein thermally treating is for a predetermined time defined by substantial elimination of the residual cross-linking element in the pressure sensitive adhesive.

32. The method of claim 22, wherein thermally treated pressure sensitive adhesive prevents decay of florescent signal of the reference dye in the sealed assay.

33. The method of claim 22, wherein the reference dye comprises 6-carboxy-X-rhodamine.

34. A method for eliminating a cross-linking element in a pressure sensitive adhesive for use in a polymerase chain reaction (PCR) system, comprising the steps of:
providing an assay support structure and a sealing member for said assay support structure;
introducing an assay into the assay support structure, the assay including a reference dye;
applying a pressure sensitive adhesive to the sealing member;
thermally treating the sealing member having the silicone-based pressure sensitive adhesive at a temperature of at least 180° C. for a period of time defined by substantial elimination of the cross-linking element;
sealing the assay within the assay support structure with the cover member; and
processing the sealed assay in the PCR system.

35. The method of claim 34, wherein thermally treating the sealing member is prior to sealing the assay within the assay support structure.

36. The method of claim 34, wherein thermally treating the sealing member is subsequent to sealing the assay within the assay support structure.

37. The method of claim 34, wherein the pressure sensitive adhesive comprises a silicone-based pressure sensitive adhesive.

38. The method of claim 34, wherein the residual cross-linking element comprises benzoyl peroxide.

39. The method of claim 37, wherein the residual cross-linking element comprises benzoyl peroxide.

40. The method of claim 34, wherein thermally treating is for a predetermined time defined by substantial elimination of the residual cross-linking element in the pressure sensitive adhesive.

41. The method of claim 34, wherein thermally treated pressure sensitive adhesive prevents decay of florescent signal of the reference dye in the sealed assay.

42. The method of claim 34, wherein the reference dye comprises 6-carboxy-X-rhodamine.

43. An assay card for use in a polymerase chain reaction system, comprising:
an assay support structure;
a cover member applicable to said assay support structure; and
a thermally treated pressure sensitive adhesive formed on said cover member, said pressure sensitive adhesive being thermally treated at a temperature of at least 180° C. for a predetermined period of time.

44. The assay card of claim 43, wherein the thermally treated pressure sensitive is free of a cross-linking component.

45. The assay card of claim 43, wherein the predetermined period of time is defined by a time required to thermally decompose a cross-linking component of the pressure sensitive adhesive.

46. The assay card of claim 43, wherein thermally treated pressure sensitive adhesive prevents signal decay of a component of the polymerase chain reaction system.
